(19) United States
(12) Patent Application Publication
(10) Pub. No.: US 2007/0207509 A1
(43) Pub. Date: Sep. 6, 2007

(54) Zinc-based screening test and kit for early diagnosis of prostate cancer

(76) Inventors: Christopher J. Frederickson, Galveston, TX (US); Leslie C. Costello, Severna Park, MD (US); Renty B. Franklin, Burtonsville, MD (US)

Correspondence Address: Benjamin Aaron Adler;ADLER & ASSOCIATES, 8011 Candle Lane, Houston, TX, 77071, US

(21) Appl. No.: 11/698,229

(22) Filed: Jan. 25, 2007

Related U.S. Application Data
(63) Continuation-in-part of application No. 10/829,732, filed on Apr. 22, 2004.
(60) Provisional application No. 60/464,510, filed on Apr. 22, 2003.

(51) Int. Cl. G01N 33/574; G01N 33/20

(52) U.S. Cl. 435/7.23; 436/81

(57) ABSTRACT

The present invention provides methods of determining if an individual is at risk for prostate cancer. The methods measures and compares free and/or bound zinc levels in a semen sample or prostatic fluid, including post massage expressed prostatic fluid, in the potential at risk individual with normal levels. A decrease in zinc level is indicative of a risk for prostate cancer.

### CROSS-REFERENCE TO RELATED APPLICATIONS

This is a continuation-in-part of non-provisional U.S. Ser. No. 10/829,732, filed Apr. 22, 2004, which claims benefit of priority of provisional U.S. Ser. No. 60/464,510, filed Apr. 22, 2003, now abandoned.

### FEDERAL FUNDING LEGEND

This invention was produced in part using funds obtained through a SBIR grant IR43CA096354-01 from the National Institutes of Health. Consequently, the federal government has certain rights in this invention.

### BACKGROUND OF THE INVENTION

1. Field of the Invention

The present invention relates generally to the field of prostate cancer diagnosis. More specifically, the present invention relates to a zinc-based screening test and kit for early diagnosis of prostate cancer.

2. Description of the Related Art

Prostate cancer kills about 40,000 men in the United States each year and there are approximately 330,000 new cases diagnosed annually. Prostate cancer is second only to lung cancer in mortality to men. Castration, treatment with anti-androgens, and prostatectomy with its associated urogenital risk, are all treatments that seriously compromise the quality of male life.

Currently, serum prostate-specific antigen (PSA), a serine protease, level and prostate digital rectal exams are the only early diagnostic tests in routine use to screen for prostate cancer. However, small, aggressive tumors can be missed by digital rectal exams and even by needle biopsy, and only modest increases in prostate-specific antigen, i.e., below the 4 ng/mL threshhold between normal and elevated PSA levels, are generated by these tumors. These aggressive tumors have the potential to suddenly dedifferentiate and grow, spread, and metastasize rapidly.

In addition to such lethal false negatives, false positives also plague the PSA test, causing unneeded tests and medical expense and distress to patients. An NCl fact sheet (1) indicates that among men above 50 years old, an age group of men most susceptible to prostate cancer, 80% of those having PSA test levels above 4 ng/mL will turn out to not have prostate cancer. The NCl Fact Sheet notes the need for a prostate cancer screen with improved ability to differentiate between prostate cancer and benign conditions such as prostatitis, benign prostatic hypertrophy (BPH) or enlarged prostate, inflammation and infection, and to differentiate between slow-growing and fast-growing cancers.

The NCl makes only a guarded recommendation for prostate cancer screening of asymptomatic men (1), and the American College of Preventive Medicine flatly recommends against it, as do some individual practitioners. Urologists generally favor screening, as does the American Cancer Society, while other groups and authors of reviews equivocate. At issue is whether the screening information leads to a clear course of action that can improve the quality or duration of life.

This controversy reflects the inadequacy of the diagnostic information obtained from the existing screening methods. Consider the patient who suffers a false negative, for example, in which a small tumor, e.g., T1a,b, T2a, is missed by digital rectal exams and missed in needle biopsy, even an ultrasound-guided, 6-sector biopsy, and does not raise the serum PSA to alarming levels, i.e. PSA below 4. Depending on the grade of tumor, a patient with a Gleason Pattern GP 4-5 tumor could have a metastatic disease with poor prognosis within a year whereas a patient with a GP 1-2 tumor might experience little changes in a year. Since most prostate cancers are slow-growing, there is a need for a routine diagnostic screen that can pick up prostate cancer earlier.

Zinc is the most ubiquitous heavy metal in the human body. In the male reproductive system semen has 3 mM zinc, which is approximately 1000-fold more than those found in saliva, tears, vaginal secretions, urine or blood. Indeed, ejaculate contains so much zinc that a zinc-sensitive dye is used routinely by police to find semen at crime scenes. Why zinc is present in semen has not been established clearly. Some researchers speculated that the zinc is an antimicrobial for cleansing the urethra. It is also true that zinc suppresses the proteolytic activity of PSA, the enzyme that cleaves seminal globular proteins to “liberate” spermatozoa, suggesting a possible zinc-mediated control process of spematozoan mobility. A role for zinc in citrate metabolism has also been noted. Finally, spermatozoa are richly endowed with zinc both in their cytosol and on their exterior, suggesting that seminal fluid might be needed to maintain a spermatozoan zinc pool.

Regardless of the function of zinc in semen, the source of zinc appears to be in part from the testes, which concentrates zinc in and on the spermatozoa, and in part from the secretory cells lining the ducts of the lateral lobes of the prostate gland. At the fine and ultrastructural level, the zinc in the prostate tubules is concentrated at the apical ends of the secretory cells, in the interstities between the cells, and most massively, in the lumen of the seminal ducts.

Physiologically, the epithelial secretory cells show relatively high velocity uptake of zinc that is driven by testosterone. Thus, one assumes that the epithelial secretory cells take up zinc, sequester it in secretory granules, and secrete the contents of the granules into the lumen, thereby generating the high zinc content of the semen (FIG. 1). The immunostaining methods developed for the zinc transporters ZnT-1, ZnT-2, and ZnT-3 fail to label the prostate epithelial cells, and thus the zinc influxing transporter has not yet been identified, although a ZIP protein has been suggested as important to this transport.

There is an overwhelming, in fact, almost unanimous, consensus from many laboratories worldwide that the prostate gland has a uniquely high zinc content which is localized to the lateral lobes and that the prostate loses from 50% to 90% of that zinc in prostate cancer. In contrast, the zinc levels increase in benign prostatic hypertrophy (BPH) and show no consistent change in prostatitis. Perhaps the best reference on the changes in zinc in the prostate in cancer and BPH is the analysis published by Zaichicks et al. who compiled data from 16 prior studies as well as their own (2).

Only one of the 17 papers reviewed failed to find decreased zinc in prostate cancer, and the other 16 all showed declines in cancer, with 15 of 16 showing ratios of diseased/control within the fairly narrow range of 0.15 and 0.55 (2). On average across the 17 studies, the zinc level was found to double in BPH with mean and median ratios 2.25 and 1.98, respectively. Other papers not covered in the Zaichick's review have also found the same basic pattern of prostate zinc changes in cancer and BPH (3-4).

Since most of the zinc in the prostate is concentrated in the lumen and secretory surfaces of the seminal tubules, e.g. in the secretory fluids, the observed drop of 50-90% in total zinc content would be expected to require a significant drop in the zinc content of the seminal fluid. This is confirmed by empirical data obtained both from patients with stage T3-T4 tumors which showed a 95% decrease in zinc in ejaculate (2), and from patients with palpable tumors which showed an 84% decrease in zinc in post-prostatic massage fluid (5). In benign prostatic hypertrophy, the zinc level was found to be either unchanged (2) or increased (5).

In contrast to the consensus findings on significant changes in zinc in prostate tissue and secretions, the literature on zinc in blood serum in prostate cancer varies between a slight decrease (6), an increase in a rat model (7) and no change (8). While disappointing from the clinical-diagnostic perspective, this is not surprising biologically. Indeed, it would be surprising if the zinc metabolism of the prostate alone could alter total body burden or serum buffering of zinc. Hence, the consensus is that zinc in blood is not a viable marker for prostate cancer.

The “ideal” prostate screening test should reliably detect even the small, nonpalpable tumors, e.g., T1a-c, T2a, that generate only modest increases in serum PSA, i.e., below 4 ng/mL, but have the potential to dedifferentiate rapidly to Gleason pattern 4-5 and thus grow and metastasize rapidly. There is a realistic chance that semen zinc measures may be a key to such an “ideal” diagnostic. After all, it is plausible that one of the first steps in prostate epithelial cell dedifferentiation would be to turn off the molecular machinery of zinc influxing. Some indirect evidence suggest this is the case (3). This would mean that semen zinc levels might be a sensitive and selective cancer indicator.

Unfortunately, measuring zinc in complex biological matrices such as semen and determining the sizes of the different “pools” of zinc and the changes, if any, in these multiple zinc pools is a daunting bioanalytic problem. Thus, the literature on zinc and prostate cancer is alarmingly error ridden. For example, estimates in the scientific literature of total zinc in prostate tissue and total zinc in semen vary over an absolute range of nearly 100-fold (2).

There is a recognized need in the art for improvements in the development and uses of state of the art bioanalytical methods to measure the distribution, speciation and concentrations of zinc in prostate tissue and seminal fluid. Specifically, the prior art is deficient in a zinc-based diagnostic test for prostate cancer. The present invention fulfills this long-standing need and desire in the art.

### SUMMARY OF THE INVENTION

The present invention is directed to a method for screening an individual at risk for prostate cancer. The method comprises obtaining a sample of a zinc-containing fluid from the individual and measuring the level of free zinc and/or zinc bound to endogenous ligands in the sample. The zinc level(s) from the at risk individual are compared with zinc levels found in a normal individual known not to have prostate cancer where a decreased zinc level in the at-risk individual compared to the level of free zinc in the normal individual correlates to a risk of developing prostate cancer, thereby screening the individual.

The present invention is directed to a related method comprising a further method step of measuring the total protein in the sample. In this related method, the measured zinc level may be a ratio of the free zinc to the total protein, a ratio of the bound zinc to the total protein, or a ratio of free zinc plus bound zinc to the total protein.

The present invention also is directed to another method for screening an individual at risk for prostate cancer. The method comprises obtaining a sample of prostate secretions in a fluid from the individual and measuring a level of free zinc in the fluid sample. The level of free zinc from the at risk individual is compared with a level of free zinc in a normal individual that does not have prostate cancer. A decreased level of free zinc in the at-risk individual compared to the level of free zinc in the normal individual correlates to a risk of developing prostate cancer, thereby screening the individual.

In related methods the prostate secretions may be in a fluid comprising seminal plasma of whole ejaculate where the obtaining step comprises separating large globular proteins and prostasomes from the seminal plasma including free zinc via size-exclusion column fractionation or a step of separating large globular proteins and prostasomes from the seminal plasma including free zinc via antibody- or aptamer-binding thereto. In an alternative related method the prostate secretions may be in prostatic fluid where the obtaining step comprises massaging the prostate to advance the prostatic fluid comprising the prostate secretions into the uretha and collecting a post prostatic massage prostatic fluid therefrom.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the proposed life cycle of zinc in prostate epithelial cells. Indirect evidence suggests that zinc pump expression may be down regulated early in cancer, thus causing reduced zinc in semen.

FIG. 2 shows four methods of staining and measuring free zinc in or on spermatozoa. Znpyr and TSQ are fluorimetric, whereas AMG gives high resolution even in the EM. Scale bar is ˜2 mm for the light micrographs.

FIG. 3 is a gel depicting the mix of proteins in seminal plasma. The gel was run at pH 4-7 with molecular weight markers of 250, 150, 100, 75, 50, 37, 25, 20, 15, and 10 KDas.

FIG. 4A shows zinc staining (brown-black) in the lateral lobes of the prostate in rat. FIG. 4B shows that zinc staining (black) fills the lumen of the lateral prostate tubule of the rat in these plastic-embedded sections. Note that the epithelial cells have only sparse grains in their apical (secretory) ends, as seen in the electron micrograph (lower panel).

FIG. 5 shows accurate zinc measurements in the left and right brain structures of 6 rats. Note the close agreement of measurements. Median L-R error is about 2% for brain regions with total zinc burdens of about 800 ng.

FIG. 6A shows zinc measurements in a genuine biological matrix (ACSF). The results show good stability and sensitivity. Note clear detection of 45 nM, which corresponds to 292 pg of total zinc. FIG. 6B shows the apoCA zinc sensing method gives a robust, ratiometric shift in fluorescence anisotropy with Zn levels in the sub-picomolar levels. FIG. 6C shows two different mutants of carbonic anhydrase having different on-rates (and have different affinities) for zinc. The fluorescence indicates zinc binding by ABDN.

FIG. 7 shows tissue distribution of total zinc (false color image) by synchrotron-induced x-ray fluorescence. This may be compared and contrasted with the image of free zinc (right panel). Both are important for understanding zinc in the prostate. Images are from rat brain; about 2 mm×2 mm.

FIG. 8 shows zinc staining of globular proteins in ejaculate. Blue fluorescence of zinc (TSQ) can be quantified whereas the black silver grains (AMG) give a higher resolution localization in the EM.

FIG. 9 depicts x-ray fluorescence spectra for three samples of prostatic fluid. Filter background is substracted.

FIG. 10 is a calibration curve for EDXRF. The blue line shows results for zinc standards, the red line shows the results obtained when using the iron as a ratiometric denominator. Both have a similar slope and a good fit to linear.

FIG. 11 is a calibration curve for the colorimetric measurement of TCA-releasable zinc.

FIG. 12 depicts the total zinc in 15 men with prostate symptoms, but either no biopsy judged necessary or biopsy-confirmed BPH.

FIG. 13 depicts the protein concentration in men presenting symptoms of prostatitis or prostate enlargement or malfunction. Two clear peaks are shown; the first, HMW, peak contains prostasomes and is identified as the “prostasomal peak”.

FIGS. 14A-14B depicts the decrease in free zinc (FIG. 14A) and protein concentration (FIG. 14B) in two men with Gleason Stage 6-8 prostate tumors (red lines). The black lines depict average results for 15 cancer-free men (+/−SD). Zinc in the prostasomal fractions (#15-20) was reduced from Abs.=0.71 to Abs.=0.32 in the two men with adenocarcinoma.

FIG. 15 demonstrates that free zinc and serum PSA do not vary with age among men 40-80 years old.

FIG. 16 demonstrates that prostatic fluid zinc is lower in cancer patients than in those with BPH. Data from EDXRF assays show that the mean and modal zinc is reduced by about ½-⅓ in men with cancer (n=15) compared to the BPH men (n=25).

FIG. 17 is the ROC analysis showing that the accuracy of detection of prostate cancer by prostatic zinc (normalized against prostatic iron) is 80%, with the Area Under the RO Curve (AUROC) is 77%.

### DETAILED DESCRIPTION OF THE INVENTION

In one embodiment of the present invention there is provided a method of screening an individual at risk for prostate cancer, comprising obtaining a sample of a zinc-containing fluid from the individual; measuring a level of one or both of free zinc and zinc bound to endogenous ligands in the sample; comparing the zinc level(s) from the at risk individual with zinc levels found in a normal individual known not to have prostate cancer; and correlating a decreased zinc level in the at-risk individual compared to the level of free zinc in the normal individual to a risk of developing prostate cancer, thereby screening the individual. In this embodiment the measured zinc level may be the free zinc in the fluid or a ratio of the free zinc to the bound zinc.

Further to this embodiment the method may comprise measuring the total protein in the sample. In this further embodiment measuring the total amount of protein in the fluid may comprise measuring ultraviolet light absorption of the protein in the sample. For example, the measured zinc level may be a ratio of the free zinc to the total protein, a ratio of the bound zinc to the total protein, or a ratio of free zinc plus bound zinc to the total protein.

Also, in these embodiments the level of free zinc in the fluid may be measured optically where the method comprises exposing the fluid to a chromophore or fluorophore each comprising a zinc-binding moiety; binding the free zinc to the zinc-binding moiety of the fluorophore or chromophore; illuminating the fluid with light; measuring the amount of light that is either absorbed by the chromophore or emitted by the fluorophore; and correlating a change in light absorption or light emission with the level of free zinc in the fluid. Representative examples of a useful chromaphor include but are not limited to dithizone, zincon, 4-(2-pyridylazo)resorcinol or other chromaphore that changes absorptive properties upon binding zinc. Representative examples of a fluorphore are fluorescein, rhodamine, allexa, or dansylamide. Representative examples of a zinc-binding moiety are quinoline, BAPTA, ethylene diamine tetra acetic acid, pyridine, or TPEN.

In one aspect of these embodiments the method further may comprise releasing the zinc bound to endogenous ligands in the fluid as free zinc and measuring a level of free zinc electrochemically before and after releasing the bound zinc. Further to this aspect the method may comprise measuring total free zinc fluorimetrically or absorptiometrically. In these aspects one example of separation is via a membrane semi-permeable to zinc. Another example of separation is by placing the fluid against a surface containing carrier or iontophore molecules effective to transport zinc ions across the surface.

In another aspect the method further may comprise releasing the zinc bound to endogenous ligands in the fluid as free zinc and measuring a level of free zinc before and after releasing the bound zinc where the free zinc is separated from the fluid comprising the endogenous ligands. In all aspects of all embodiments the zinc-containing fluid may comprise whole ejaculate, whole seminal fluid, seminal plasma, or prostatic fluid.

In another embodiment of the present invention there is provided a method for screening an individual at risk for prostate cancer, comprising obtaining a sample of prostate secretions in a fluid from the individual; measuring a level of free zinc in the fluid sample; comparing the level of free zinc from the at risk individual with a level of free zinc in a normal individual that does not have prostate cancer; and correlating a decreased level of free zinc in the at-risk individual compared to the level of free zinc in the normal individual to a risk of developing prostate cancer, thereby screening the individual.

In one aspect of this embodiment the prostate secretions may be in a fluid comprising seminal plasma of whole ejaculate where the step of obtaining comprises separating large globular proteins and prostasomes from the seminal plasma including free zinc via size-exclusion column fractionation. In a related aspect the prostate secretions may be in a fluid comprising seminal plasma of whole ejaculate where the step of obtaining comprises separating large globular proteins and prostasomes from the seminal plasma including free zinc via antibody- or aptamer-binding thereto.

In another aspect of this embodiment the prostate secretions are in prostatic fluid, the step of obtaining may comprise massaging the prostate to advance the prostatic fluid comprising the prostate secretions into the urethra and collecting a post prostatic massage prostatic fluid therefrom. In this aspect the prostatic fluid may be collected from the urethra in a first volume of urine produced post prostatic massage. Further to this aspect the method comprises repeating massage until the prostatic fluid emerges from the urethra and collecting the post prostatic massage prostatic fluid onto a surface.

In this embodiment the free zinc in the prostatic fluid may be measured fluorimetrically using a fluorophore comprising a zinc-binding moiety. Examples of the fluorophore and zinc-binding moiety are as described supra. Further to this embodiment the method may comprise adding detergent to the prostatic fluid and lysing and dissociating prostasomes and globular proteins in the prostatic fluid to release zinc bound thereto as free zinc, where free zinc is measured before and after lysing. In another further embodiment the method may comprise mixing the fluorophore with the prostatic fluid containing the free zinc.

In yet another further embodiment the method may comprise attaching the fluorophore at a distance no more than 350 nm from a surface of a solid substrate to which the prostatic fluid containing the free zinc is exposed. In one aspect of this further embodiment the method further may comprise exciting the fluorophore with an evanescent wave of light that is totally internally reflected within the solid substrate and detecting the light emissions of the excited fluorophore to measure the free zinc. In another aspect the method may comprise positioning a sensor on a surface of the solid substrate opposite to the surface exposed to the prostatic fluid to detect fluorescent emissions of a light excited fluorophore to measure the free zinc. In yet another aspect the method further may comprise separating the prostatic fluid from the fluorophore via a semipermeable membrane permeable to zinc ions but not permeable to the fluorophore.

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein, the term “subject” refers to any recipient of the prostate cancer screening as discussed herein. Preferably, the subject is a mammal, more preferably the subject is a human. As used herein, the term “free zinc” refers to rapidly exchangeable zinc which is that concentration of zinc that will bind to saturation to a zinc-binding sensor molecule having moderate affinity (KD˜1 nM) and a diffusion-limited on-rate within a brief epoch, e.g., 1 min, after mixing. Thus, “free zinc” is the zinc one can “see” with a calorimetric, voltametric, or fluorescent sensor within 1 minute. Thus, the term “free” is defined completely by the off-rate of the ligand with which the zinc is associated prior to measuring. If the zinc is Zn2+ coordinated with Cl— or acetic acid, the “off rate” is virtually instantaneous. With weak-binding organic ligands, such as citrate (KD˜5) or glutamate (KD˜6), the off rates are still rapid (msec to sec), but for tightly-binding ligands, such as carbonic anhydrase, the time for one-half of the zinc to come off spontaneously is about 2 years.

The present invention provides analytical tools for measuring the amount and speciation of zinc in semen or prostatic fluid. These tools facilitate basic research and provide for a zinc-based diagnostic kit for prostate cancer. It is contemplated that a measurement of semen and/or prostatic fluid zinc levels, which fall in cancer, alone or combined with serum PSA levels, which rise in cancer, could provide sensitive and selective early diagnosis for prostate cancer. Test kits for routine testing of seminal or prostatic zinc in the clinic or seminal zinc at home can be developed.

A valid zinc diagnostic test for prostate cancer requires speciation of semen zinc in that the fall in semen zinc at the onset of prostate cancer is not equally specific to the different semen zinc pools, i.e. free zinc, zinc bound to endogenous ligands, such as microligand bound zinc, small protein bound zinc, large protein bound zinc, and spermatozoan zinc. Identification of the semen zinc pool(s) which change earliest and most consistently at the onset of prostate cancer is necessary to optimize the sensitivity and selectivity of the prostate screening test.

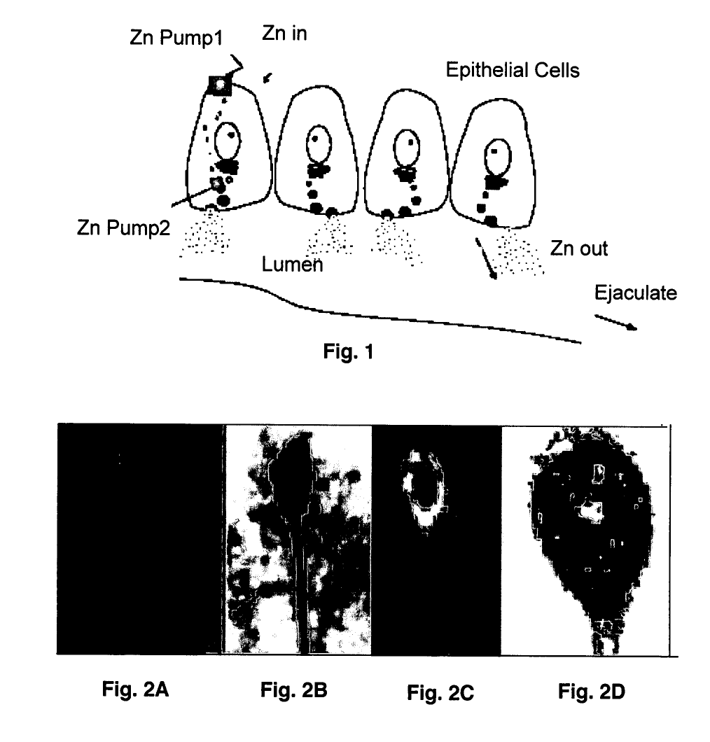

For the present invention, a key observation is that zinc content of semen and prostate tissue apparently drops rapidly in the earliest stages of prostate cancer (2). This fall in zinc may be due to down-regulation of the zinc influxing pumps of prostate cells in the earliest stages of endothelial cell dedifferentiation and proliferation (FIG. 1). Whatever the mechanism might be, the diagnostic value of this metabolic change could be life-saving.

For a zinc-based cancer diagnostic, one must determine exactly how much zinc is in the ejaculate or in post-prostate massage expressed prostatic fluid, how it is distributed among which groups of endogenous ligands, what the within and between individual variability is and, of course, which of the zinc pools will offer the best early diagnostic for cancer. Thus, the crucial steps in the development of zinc-based prostate cancer screening method and diagnostic kit include elucidating exactly how much zinc is really in ejaculate or expressed prostatic fluid, how zinc is distributed among the many quantitatively-important pools, such as, but not limited to, free zinc, zinc bound to endogenous ligands, such as microligand-bound zinc, small protein bound zinc, and large protein bound, and which of those pools changes earliest and most consistently at cancer onset.

The present invention develops and provides state of the art procedures for measuring the distribution, speciation and concentrations of zinc in prostate tissue and seminal fluid, i.e., ejaculate or the post-prostate massage expressed prostatic fluid. Measures to be determined include: free versus bound zinc in seminal plasma or prostatic fluid; ligand binding, i.e., speciation, of zinc in semen; free versus bound zinc in prostate tissue; zinc concentrations in individual spermatozoa; and histochemical localization(s) of the free stainable zinc pool using Timm-Danscher fluorescence and Synchrotron X-ray fluorescence.

Using the methods developed herein, the means, ranges, and variances of zinc contents in prostate tissue, prostatic fluid and ejaculate can be determined in men with or without, as a control, prostate cancer. Methodology was developed to allow determination of: 1) free and total zinc in whole seminal fluid or ejaculate; 2) free and total zinc in seminal plasma; 3) free and total zinc in prostatic fluid; 4) zinc bound to specific subsets of seminal proteins; and 5) zinc concentration in individual spermatozoa.

Provided herein is a method of screening for prostate cancer by measuring the amount of zinc in semen samples. Decreased levels of zinc compared to those found in normal individual would indicate such individual is at risk of developing prostate cancer. The semen samples can be whole seminal fluid, seminal plasma, expressed prostatic fluid, spermatozoa, cytosol of spermatozoa or seminal globulin protein.

Specifically, there are three measures of zinc in semen with diagnostic potential: (i) the concentration of “free” or “rapidly-exchangeable” zinc in the semen, (ii) the concentration of zinc bound to organic ligands in the semen, such as proteins, peptides, amino acids, small molecules, and (iii) the zinc in cells, such as spermatozoa or endothelial cells that have sloughed into the semen. FIG. 2 summarizes the three main methods of localizing and/or quantitating weakly-bound zinc. These histochemical methods have different strengths and different uses.

It is contemplated that free zinc also may be measured in prostate secretions present in prostatic fluid. For example, and without being limiting, prostate secretions may be obtained by prostate massage to channel or advance the prostatic fluid to the urethra to collect it therefrom. Particularly, the prostatic fluid may be collected in a first volume of urine produced post massage. Alternatively, upon further prostate massage the prostatic fluid will emerge from the uretha whereupon it may be collected onto an appropriate surface. The free zinc may be measured by fluorometric methods known in the art or by methods described herein.

Free zinc or total zinc, including bound zinc released as free zinc, whether seminal or prostatic, may be measured optically by exposing a free zinc-containing fluid to a chromophore or fluorophore in a colorimetric, absorptionmetric or fluoremetric assay. Zinc-binding moieties present on the fluorophores or chromophors, such as, but not limited to, quinoline, BAPTA, ethylene diamine tetra acetic acid, pyridine, or TPEN, bind free zinc from the fluid. Upon illumination, the amount of light absorbed by the chromophore or emitted by the fluorophore positively correlates to the amount of free zinc in the fluid. Chromophores, such as dithizone, zincon, 4-(2-pyridylazo)resorcinol or other molecule that changes absorptive properties upon binding zinc, and fluorophores, such as fluorescein, rhodamine, allexa, or dansylamide, are well known in the art and commercially available.

More particularly, a fluorophore may be mixed with the zinc-containing prostatic fluid. Also, the fluorophore may be attached to a surface of a solid substrate, to which the prostatic fluid is exposed, at a distance of no more than 350 nm therefrom. The attached fluorophore may be excited with an evanescent wave of light which is totally internally refelected within the solid substrate and emitted light used to measure the free zinc. Alternatively, a sensor may be positioned on the surface that is opposite to the surface exposed to the prostatic fluid to detect light emitted by the fluorophore and thereby measure the free zinc.

Generally, the fluorescent methods are best for quantitation as they are stoichiometric and with the apoCA versions ratiometric. Thus, for the purpose of measuring the weakly bound zinc: the present invention uses fluorescence analysis. Among the fluorescence methods, there are further choices based on the subcellular location of the zinc to be measured. For example, the membrane impermeable apoCA will not label zinc in vesicles, nor will the “trappable” Newport green, which is metabolized in cytosol, label zinc in the cytosol. In contrast, the lipophilic stains TSQ and Zinpyr will stain zinc in intracellular organelles, cytosol, and in extracellular fluid.

Generally, free and total zinc in solution can be measured by apoCA fluorimetric method and stable isotope dilution mass spectrometry, respectively. Alternatively, microspectro-fluorimetric methods or silver staining autometalography can be used to measured zinc that is not in solution. Thus, extracellular zinc, such as zinc on the outer surfaces of spermatozoa or zinc loosely coordinated with globular proteins, can be stained with cell-impermeable stains such as Newport Green, and the fluorescein-based metal sensors Zinpyr or Zin-naphthopyr (ZNP) (9), or by TSQ. U.S. Patent Application No. 20020106697 discloses ZP-4 and ZP-8 as examples of Zinpyrs.

Moreover, the instant zinc-based screening method can be combined with PSA assays currently in use to obtain screening with enhanced accuracy. Results of decreased levels of zinc combined with increased levels of PSA compared to those found in normal individual would render prostate cancer screening more sensitive and accurate. This provides corroboration of results with a higher level of control for the screening method

A simple, inexpensive test kit with calorimetric, or even, given the $5 LED's and CCD'S on the market, a ratiometric fluorimetric measurement system is contemplated. The test may be performed in a clinic for measuring the clinically-appropriate “pool” of semen or prostatic fluid zinc. Additionally, the kit also may be a home-use test kit that would measure the amount of zinc in whole seminal fluid. Together with the information from serum PSA, for which home-use test kits are already on the market, the information from the semen zinc test could give men a new degree of certainty about the health of their prostate glands.

The kit measures zinc in one or more pools of free zinc, bound zinc or zinc in cells, as described above. Diagnosis may be based on the relative abundance of zinc in these pools and depends upon which of these pools sizes or ratios of zinc abundance in different pools is the most accurate predictor of nascent prostate cancer.

The method of measuring free zinc is to separate the free zinc from the whole semen by dialysis. Dialysis membranes with pore size of 100 MW have been shown to allow zinc to diffuse from biological fluids, while keeping fluorescent probes for zinc restrained. In the kit for free zinc, a fluorescent probe for zinc is placed on one side of a dialysis membrane or molecular sieve and the semen is placed on the other and time is allowed for the zinc to diffuse through and bind to the fluorescent probe. In addition to the dialysis step, treatment of the sample with a detergent, e.g. triton-X 100, to lyse the membranes of prostasomes can also be employed. This is because some amount of the zinc secreted by prostate epithelial cells into the semen may be sequestered in secretory prostasomes.

Many probes are known in the art. For example, a probe may be, inter alia, apoCA+a reporter, such as dansylamide or ABDN, a Zinpyr dye or stain, such as ZP-1, ZP-4 or ZP-8, a zin-napthopyr, such as ZNP-1, TSQ, Fluo-zinc, or coumazin. Others of such probes are known and readily available and can be used for this measurement.

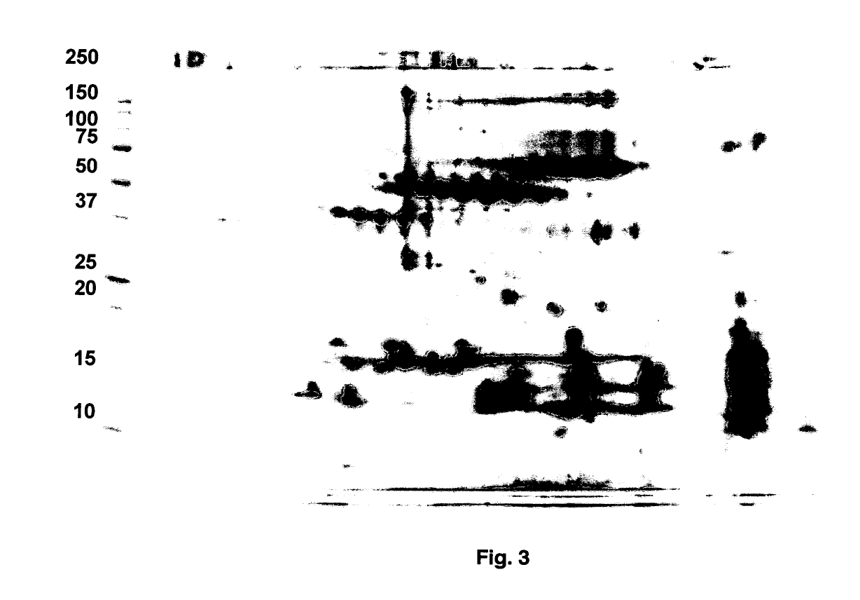

To measure the bound zinc, additional steps of sample preparation are required. First, the zinc-binding ligands must be separated to isolate one or more of the ligands with the to-be-measured zinc. A sample of the mix of proteins that is in seminal plasma is shown in FIG. 3. To separate out from this mix of proteins, or other zinc-binding organic molecules, standard separation methods familiar to those skilled in the art are used. Such methods may be chromatography, gel separation and antibody-based extraction/purification. Not all zinc-binding ligands need to be identified or purified.

A simple immobilized antibody or aptamer can be used to trap the zinc-binding ligand of interest on a substrate, with simple washing used to remove the non-selected molecules and vehicle from the substrate. To measure the concentration of zinc in the isolated zinc-binding ligands, the zinc is first released by treating captured material with an agent such as nitric oxide, hydrogen peroxide or weak acid, or other chemical treatment methods known to those skilled in the art to release the zinc from organic ligands, to denature the zinc-binding motif thus causing the zinc to be released into the surrounding fluid. The free zinc can then be determined by the same fluorimetric methods described above. Thus the kit may further contain an antibody immobilized upon an appropriate substrate to separate zinc-binding ligands.

To measure the total zinc in cells, the cells are separated from the seminal plasma, e.g. by simple filtering. The separated cells are both lysed by triton X, as described, and bound zinc is released by the method described above. The resulting free zinc is measured by the fluorimetric methods described above.

In kit form, all of the steps above can be accomplished on simple, take-home formats, such as those utilized for measuring various analytes, e.g., glucose, cholesterol, or drugs of abuse, in bodily fluid, such as serum or urine, at home. Antibody separation is used in kits like home pregnancy tests, colorimetric tests are used in glucose, cholesterol, ketone, and other home-tests, and filtration of material including cells and cell debris out of fluid is routine in home-tests systems, e.g. in glucose test kits.

One example of such a kit comprises a “ZnDectec” cassette, a pouch comprising a dialysis bag, a small digital reader and a chart. The “ZnDectec” cassette may be a 4-5 cm container comprising a mixture of carbonic anhydrase (apoCA) and a reporter molecule, such as dansylamide (DNSA), as described. In using this kit whole seminal fluid is placed into the pouch which is designed to fit into the cassette. The free zinc ions in the sample pouch will move freely out of the pouch and into the detection cassette where the zinc ions will bind strongly to the apoCA and form the holoCA-dansylamide complex.

The pouch which is substantially depleted of free zinc ions is then removed from the cassette. The cassette is placed into a simple fluorescence reader having excitation and emission filters set to collect the fluorescence of the holoCA-dansylamide complex, but not that of DNSA or apoCA-DNSA. The fluorescence reader will convert the fluorescence values to values of zinc levels. An individual can check the chart included in the kit against the values of zinc levels obtained and determine whether the measured zinc levels fall into one of three ranges: normal, pre-disposition to prostate cancer and prostate cancer.

Beyond semen testing, zinc changes may be used as a basis for differential imaging of healthy versus cancerous prostate tissue. There are many non-toxic or benign zinc binding compounds, including such citrate, histidine, diethyldithiocarbamate, such as used in Antabuse, and clioquinol which is a USP antimicrobial, that can be taken orally and reach the prostate tissue. To image zinc, a molecule or agent which undergoes a distinctive shift in a parameter like infrared light absorption or NMR resonance frequency upon binding zinc is required. Such a zinc contrast agent would allow imaging of the healthy prostate by optoacoustic imaging or MRI. NMR contrast agents for zinc have already been demonstrated (10). Imaging of the prostate by ⁶⁹Zn or ⁷²Zn ultra-short lived nuclides has also been suggested and could be made to work with contemporary instruments (11).

The following examples are given for the purpose of illustrating various embodiments of the invention and are not meant to limit the present invention in any fashion.

### EXAMPLE 1

Subcellular and Ultrastructural Localization of Zinc

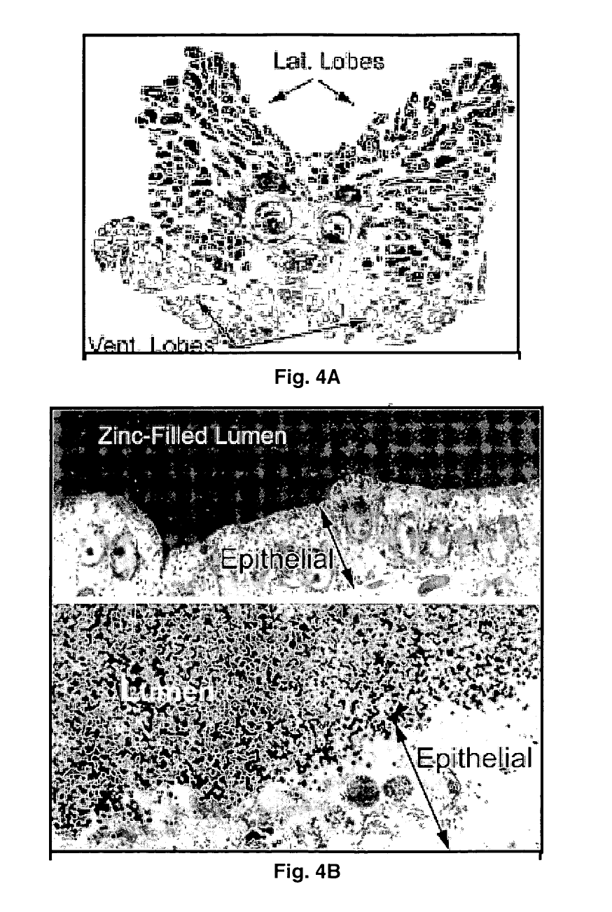

Though it is not a quantitative method, the silver AMG methods of Danscher (12) are the definitive method for determining the fine and ultrastructural localization of free or weakly-bound zinc pools. In skilled hands, these methods show as few as 10 atoms of zinc (13). In the male reproductive system, it has been shown that zinc is gradually added to the spermatazoa as they mature through the epididymis and remains hugely enriched through the spermatazoan trip into ejaculate. FIGS. 4A and 4B show enormous amounts of zinc in the prostatic secretions within the tubules. Further, the electron microscopic view shows that the zinc is selectively concentrated in apparent secretory packets in the epithelial cells, poised, as it were, to be secreted into the zinc-rich lumen.

### EXAMPLE 2

Distribution of Total Zinc in Tissue by X-Ray Fluorescence

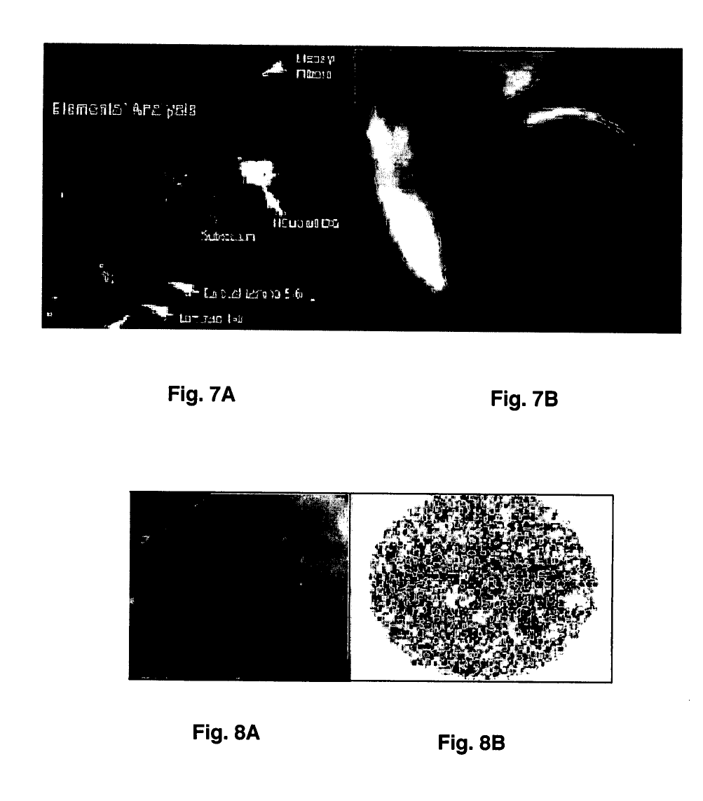

To determine the distribution of zinc among different cells, globular proteins or different regions of tissue, zinc imaging with synchrotron-induced X-ray fluorescence is used (FIG. 7). This technique can be used to determine the distribution of zinc in the different regions of the prostate gland and in different components, such as globular proteins and spermatozoa, of dried whole ejaculate or prostatic fluid as described below.

### EXAMPLE 3

Measurement of Zinc Using apoCA-ABDN Via Fluorescence Ratiometric Methods

Analysis of Free Zinc

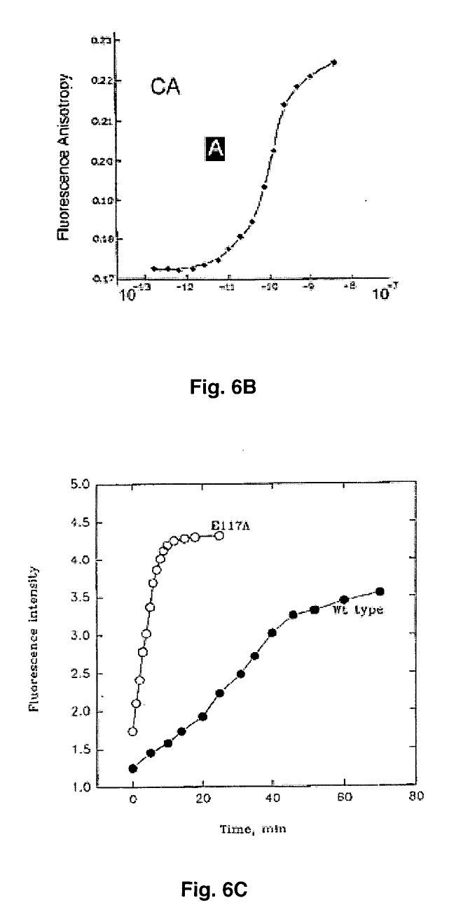

The present invention employs carbonic anhydrase (CA) as the zinc detector and either ABDN or dansylamide as the fluorescent reporter for high-accuracy measurement. In operation, the fluorescent reporter binds to the CA if and only if the CA has a zinc in the “pocket”, i.e., holoCA. Upon binding to the holoCA, the reporter undergoes an increase in intensity and blue-shift in wavelength of the emission (FIG. 6A), as well as a change in fluorescence anisotropy (FIG. 6B). By starting with the apoCA, one then adds a test solution, and monitors the fraction of the reporter that is blue-shifted, or anisotropy-shifted, by the occurrence of zinc binding to the apoCA (FIG. 6A). The wavelength and anisotropy ratio measurements can be done in test tube or by confocal microscope.

An entire family of genetically-engineered CA proteins with different affinities for zinc can be generated (FIG. 6C). By simply performing a competition assay with these different CA mutants, the binding strength of zinc to different ligands in ejaculate can be measured.

Method for Fractionation of Semen Components

All containers, reagents and materials were cleaned of zinc by ion exchange, soaking in hot EDTA which chelates Zn2+ or hot acid, multiple rinses in 18 Mohm water, as appropriate. The success of all cleaning methods was verified by testing each procedure for the “blank” zinc contaminant level. Surfaces, e.g. soft glass, which are known to adsorb or release large amounts of zinc from solution are avoided.

Fresh ejaculate collected in tubes certified to neither adsorb zinc from samples nor to contaminate them within the limits of detection, i.e., femtogram, 10⁻¹⁵ g, was incubated at room temperature for 20 minutes to allow liquefaction. The samples were then diluted with one volume of 200 mM sucrose, 2.4 mM MgCl₂ and centrifuged at 400×g to remove intact sperm cells (18). The supernatant was stored at −80° C. for subsequent analysis, with freeze-thaw damage to proteins minimized.

Seminal plasma proteins were separated by size exclusion chromatography (19) run at 4° C. The seminal plasma samples were diluted to a protein concentration of 1 mg/mL in 150 mM NaCl, 100 mM sodium phosphate buffer (pH 7.1, buffer A) and up to 5 mL was then filtered through a 0.45 um low protein-binding filter. The diluted seminal plasma samples (2-3 mL) were then applied to a 30 cm Sephacryl S300 HR column having a resolution range of 10 to 1500 kDa (Amersham Pharmacia Biotech). The mobile phase was buffer A, delivered at a flow rate of 1 mL/min via a peristaltic pump (Gilson) and 1 mL fractions were collected. Total protein in the eluted fractions was determined spectrophotometrically by 214 nm absorbance.

Total zinc content, i.e., free plus bound, of each semen component fraction was then determined by stable isotope dilution mass spectrometry and free zinc was determined by the recently developed apoCA fluorimetric method (16). The latter method for free zinc is a fluorescence ratiometric method in which a fluorescent reporter molecule such as ABDN binds to a zinc sensor molecule, the metalloenzyme carbonic anhydrase, CA, if and only if the CA has a zinc in the “pocket.” The zinc-containing holoenzyme increases the fluorescence of the reporter.

ApoCA was prepared by removing the Zn²⁺ with dipicolinate and dialysis against a zinc chelator. The apoCA was then mixed with the fluorescent reporter, both at 2 mM, in 50 mM HEPES-buffer. When there is no Zn²⁺ in the fraction, i.e., less than the femtogram detection limit, the apoCA remains without zinc and does not bind to the fluorescent reporter, which emits its native fluorescence. When Zn²⁺ is present in the fraction, it binds stoichiometrically to the CA (K_{D} of 4 μM).

The resulting holoCA binds to the reporter, causing a shift in its emission wavelength from 600 nm to 560 nm and an 8-fold increase in emission intensity. This system readily measures zinc in fluids from μM levels up. For Zn²⁺ levels well above the K_{D}, for example, low μM levels, the percent-occupancy approach is used in which the upper limit of the fluorescence sensitivity is set by the concentration of apoCA used and the lower limit is about 1% of that. For example, with 100 μM of apoCA and 100 μM of ABDN, the fluorescence shift will be maximal at 100 μM Zn²⁺ and is just detectable at about 0.1 to 1.0 μM.

The chromatography column is calibrated regularly with molecular weight standards (Sigma) and a parallel, calibrated column is used to resolve zinc-containing CA II (Sigma) to demonstrate efficacy of fractional zinc determination. Because carbonic anhydrase is the basis for the free zinc assay, the use of carbonic anhydrase holoenzyme with zinc and carbonic anhydrase apoenzyme with zinc removed provides an internal reference for total zinc as a fraction of total protein.

Measurement of Zinc in Fluids

To measure zinc in a particular fluid, such as the whole semen plasma or prostatic fluid, including post prostate massage expressed prostatic fluid, one starts with an apoCA-ABDN solution at 10 times the expected zinc concentration. An aliquot of plasma is added and a fluorescence spectrum is obtained. The magnitude of the emission peak shift relative to a control sample is observed. By appropriate dilution of the unknown, one then brings the sample into the right zinc concentration range for the final spectrum.

Calibration curves are run by the method of standard additions, using the matrix, e.g. seminal plasma, as the vehicle and adding zinc. Zinc chelators such as CaEDTA are used to quench the fluorescence in order to verify that the emission shift is indeed due to zinc. SIDMS verifies the final concentration of zinc bound to the carbonic anhydrase after the carbonic anhydrase is isolated by dialysis, providing a final verification of the absolute accuracy of the method.

Access to an entire family of genetically engineered carbonic anhydrase proteins having a range of affinities for zinc would allow measurement of the binding strength of zinc to different ligands in ejaculate by simply competing for the zinc with the different carbonic anhydrase mutants (20-21).

### EXAMPLE 4

Measurement of Free Zinc that is Not in Solution

To measure free zinc in material that is not in solution, such as in the cytosol of individual spermatozoa or in seminal globular proteins, microspectrofluorimetric methods for measuring zinc in brain tissue were used (16, 22). In this method, the material is stained to show the zinc pool of interest. Extracellular zinc, such as zinc on the outer surfaces of spermatozoa or zinc loosely coordinated with globular proteins, can be stained with either cell-impermeable Newport Green, or by TSQ (FIG. 8). Each stain has its particular strengths and weaknesses in this application. The material is stained, smeared on slides and the fluorescence is quantified in a fluorescence microscope and quantitative images captured on a laser-scanned confocal instrument and a cooled CCD camera.

The distribution of total zinc in the different regions of the prostate gland and in different components, e.g., globular proteins and spermatozoa, of dried whole ejaculate can be determined by Synchrotron-induced X-ray fluorescence of zinc (23). The distribution of free zinc can be determined by histoanalytical methods specific to the subcellular localization of the zinc (12).

### EXAMPLE 5

Methods for Measuring Total Zinc in Prostatic Fluid

Stable Isotope Dilution Mass Spectrometry (SIDMS)

Ejaculate, zinc-containing tissue or other samples, e.g., but not limited to, seminal plasma, prostatic fluid, including post prostate massage expressed prostatic fluid, or specific protein fractions are collected in tubes certified to neither remove zinc from samples by absorption or adsorption nor contaminate the samples within the limits of detection. Because semen has about 1000-fold more zinc than any other biological fluid, contamination will be less of a problem than usual in this type of work.

The samples are spiked with a precisely measured amount of ⁶⁴Zn or ⁶⁶Zn before subjected to dissolution procedures to reduce them to elemental composition. The SIDMS sample preparation room is a class-100 clean room within which personnel wear clean-room over-garments and hair covers. All reagents are double-distilled in the laboratory in quartz stills, and made using ultrapure grade materials and 18 MOhm or better grade de-ionized water. Critical sample contact surfaces are all TFE teflon, polypropylene or quartz. For small sample determinations, it has been previously established that the error variance of the whole-process blank for zinc is no greater than ˜2 ng S.D. (14).

Sample preparation after spiking generally progresses by (i) lyophilization; (ii) weighing; (iii) dissolution to elemental composition in concentrated hot nitric acid or perchloric; (iv) purification of zinc by ion exchange; (v) determination of ^{66/64}Zn ratio in the Isotope ratio Mass Spectrometer; and (vi) calculation of initial zinc concentration in the sample.

The accuracy of the final measure of zinc concentration, however, does not depend on the instrument accuracy, but upon the degree of contamination or loss of zinc during sample preparation. This “blank” amount of zinc in the present SIDMS micromethods has been lowered to a SD of +/−0.9 ng for n=5 (14-15). Thus, in order to obtain a coefficient of variance of 5%, a reasonable standard, a minimum of 18 ng of zinc per sample is required. Given that all soft tissue has at least 60 ppm (dry) of zinc, this means no more than about 300 ug of tissue needed to be analyzed for 5% coefficient of variance.

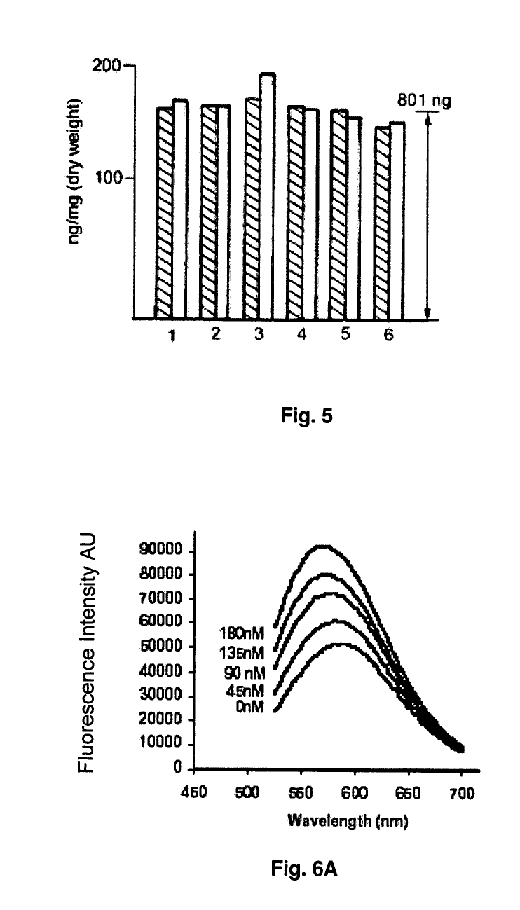

FIG. 5 depicts an example of this kind of accuracy. The left and right brain regions from individual rats were shown to vary by no more that a few percent, or in absolute terms by no more that a few nanograms.

Flame Atomic Absorption Spectrophotometry (AAS)

Analyses of total zinc are performed in duplicate using AAS (PerkinElmer 5100 instrument). For sample preparation 10 μl-aliquots of semen plasma supernatant are mixed with 2990 μl of 0.5 M HNO₃ (OmniTrace Ultra, Merck) and incubated in closed test tubes for ˜2 h at 60° C. Operating parameters are air/acetylene flame, 213.9 nm zinc line with deuterium lamp background correction. Zinc standards (Sigma-Aldrich) are 1000 mg/l and are diluted in 0.5 M HNO₃. Calibration curves down to the range of 0.05 μg are routinely obtained during sample analysis and are quite linear.

Energy Dispersive X-Ray Fluorescence (EDXRF)

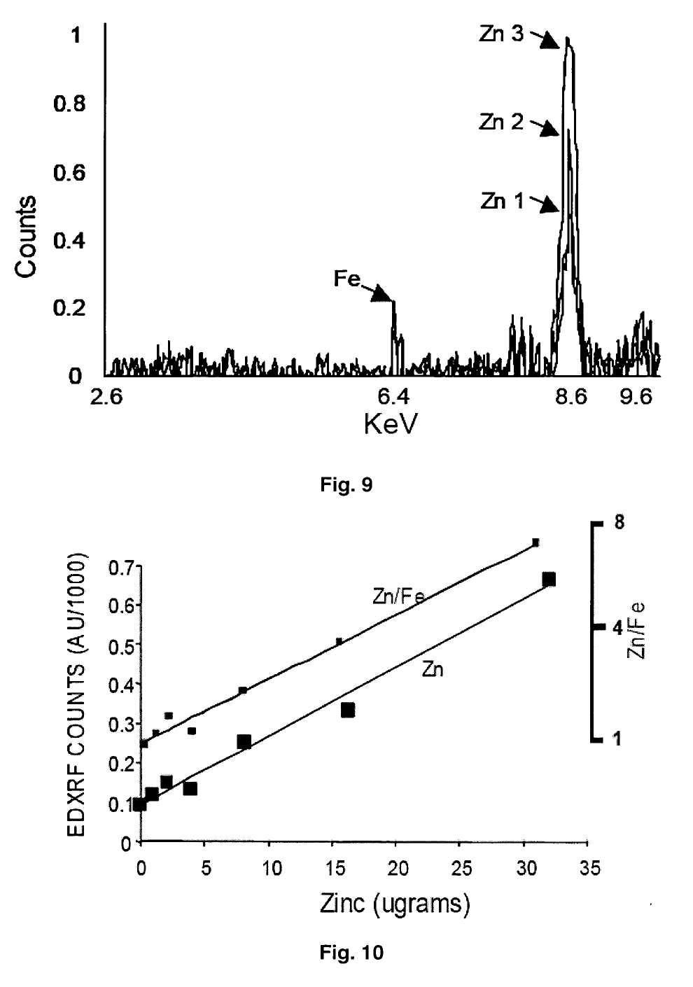

For analysis of total zinc by EDXRF 10 μl of fluid is withdrawn from a subject's sample container, e.g., an eppendorf tube, with a precision pipette and spotted down onto a forensic filter. The filters are then assayed in the thin filter mode by placing the filters over the detection window of the EDXRF analyzer. Each filter is analyzed in duplicate for 2 min each. Blank filters serve as background controls. FIG. 9 shows that the Fe peaks (6.4 KeV) all suprerimpose, whereas the zinc peaks (8.6 KeV) vary by more than 100% from lowest to highest. It is contemplated that this method is useful as a high-volume clinical test in which multiple samples are placed into wells in a 96- or 256-well format plate and all scanned by EDXRF with a motor-driven stage moving the samples across the ˜1 cm² window

Ratiometric Method Demonstrating Zinc Concentration is Independent of Volume

Several solutions of different concentrations of ZnCl2 were prepared containing a constant concentration of Fe (2 μg/ml). Two volumes, either 100 μl or 200 μl of ZnCl2 solution were spotted on filter paper, dried and the filters were analyzed as described. Results showed that the limit of detection for zinc under these conditions was ˜2 μg zinc. To eliminate any unknown effects of the instrument's software calculation of zinc concentration expressed as μg/cm2, all data was recorded as counts per second (cps) and a standard curve for zinc was used to calculate zinc concentration when required. The total mass of zinc on the filter could be determined independently of the volume. FIG. 10 shows that when the amount of zinc is increased by increasing the volume (2×) of the same zinc concentration, the relationship between zinc mass and cps is linear. Also, when the ratio of Zn/Fe (counts/counts) is plotted against zinc amount, the relationship also is linear.

Absolute Zinc Mass Method

Alternatively, as prostatic fluid is contained in an eppendorf tube, an aliquot of an exact volume of fluid may be placed onto the filter. This eliminates the need to use the Zn/Fe ratio to determine concentration of zinc (μg/μl) in the prostatic fluid. Fe is recorded, however, so that ratiometric data is available.

Colorimetric Procedure for “TCA-Releasable Zinc”

The zinc-indicator, 2-(5-bromo-2-pyridylazo-)-5-(N-n-propyl-N-3-sulfopropylamino)phenol (5-BrPAPS) is used as a simple, quick and accurate calorimetric indicator. The test is suitable for a multi-well plate format or using a color stick. 5-BrPAPS has a Kd for zinc binding in the low micromolar range and the Zn:5BrPAPS complex absorbs strongly (ex coeff˜120,000) at 560 nm. This method is easily linear across the zinc concentration range of interest and generates a vivid, reddish signal in samples that are in the normal range, with only pale yellow in the range of the typical prostate cancer subject. This is considered a measure of the “TCA-releasable zinc” because it is contemplated that treatments will presumably cause some of the zinc ions in the prostatic fluid to be released into the aqueous phase as free zinc whereas other zinc ions, e.g., those bound into zinc-finger motifs, to precipitate still co-ordinated to the denatured proteins that wind up discarded in the pellet.

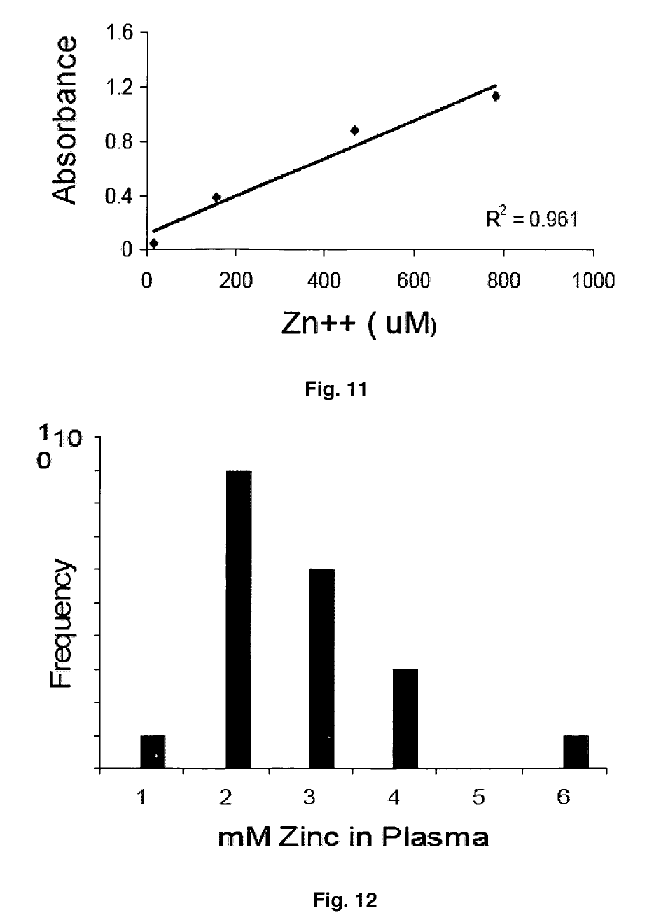

To perform the assay zinc is released from proteins and the proteins are precipatated. A 20 μl sample of prostatic fluid is diluted with 300 μl 75% TCA in a 2 ml microfuge tube. The contents are vortexed and centrifuged. The pellet is removed. A 125 μl aliquot is transferred to a new microfuge tube followed by the addition of 1000 μl 5BrPAPS and 250 μl salicylaldoxime reagent in 10 mM HEPES at pH 7.0. The prostatic fluid samples, zinc standards and blank are then run simultaneously and compared by absorption at 560 nm to measure the color change. A calibration curve is shown in FIG. 11.

### EXAMPLE 6

Free and Total Zinc Analysis in Prostatic Fluid

Normal Distribution

Frozen human semen from 3 young men (sperm donors) and from 15 men with prostate symptoms, but either no biopsy judged necessary or biopsy-confirmed BPH (FIG. 12) was liquefied at 37° C. for 30 min. The samples were centrifuged at 1000×g for 10 min to separate spermatozoa from the seminal plasma. Free zinc was measured spectrophotmetrically in the seminal plasma by adding 1011 of seminal plasma to 990 μl of Zincon (extinction coefficient of the Zn:Zincon at 620 nm; 17,500 M⁻¹ cm⁻¹). This procedure gave a working range of approximately 1 μM to 100 μM in the stoichiometric assay mode. To measure total zinc by FAA, 10 μl of seminal plasma samples were diluted into 1810 μl of 0.5 M HNO3 and analyzed for total zinc by standard methods. Two measurements were made for each sample.

Total zinc in the seminal plasma was about 3.5 mM (range 3-6 mM). The concentration of free zinc averaged about 0.4-0.5 M. The 0.4 mM value of free zinc is about 400,000-fold higher than that found in most extracellular fluids and the 3.5 mM value of total zinc is about 20-fold higher than most soft tissue. Thus, it will not be analytically difficult to detect the changes in free zinc in semen as these levels are not trace amounts.

Distribution of Free and Total Zinc Among the Pools of Zinc in Ejaculate and Seminal Plasma

17 men aged 42 and older and presenting symptoms of prostatitis or prostate enlargement or malfunction provided ejaculate samples collected at home. A sample kit with a unique identification number consisted of a collection vial, cold shipment container and instructions for collection of the ejaculate sample at home. The unique identification number was used to identify the samples and to correlate the data obtained with pertinent information regarding the participant's prostate health.

Sample preparation was as described for those obtained from normal men except that the 200 μl of the seminal plasma was subjected to size-exclusion fractionation into 42 fracions (500 μl) on a Sephadex 0 column and the free zinc and protein concentration were then analyzed for each fraction. Free zinc was measured after dilution of 10 μl of each fraction into 90 μl of Zincon solution, as described above. Total protein and peptide concentrations were measured with a micro BCA protein assay kit (Pierce Biotechnology). 20 μl of each seminal plasma aliquot was mixed with 280 μl of 20 mM Tris-Hcl buffer, pH 7.4 and 200 μl of assay reagent. The solutions were incubated at 60° C. and absorbance was measured at 562 nm.

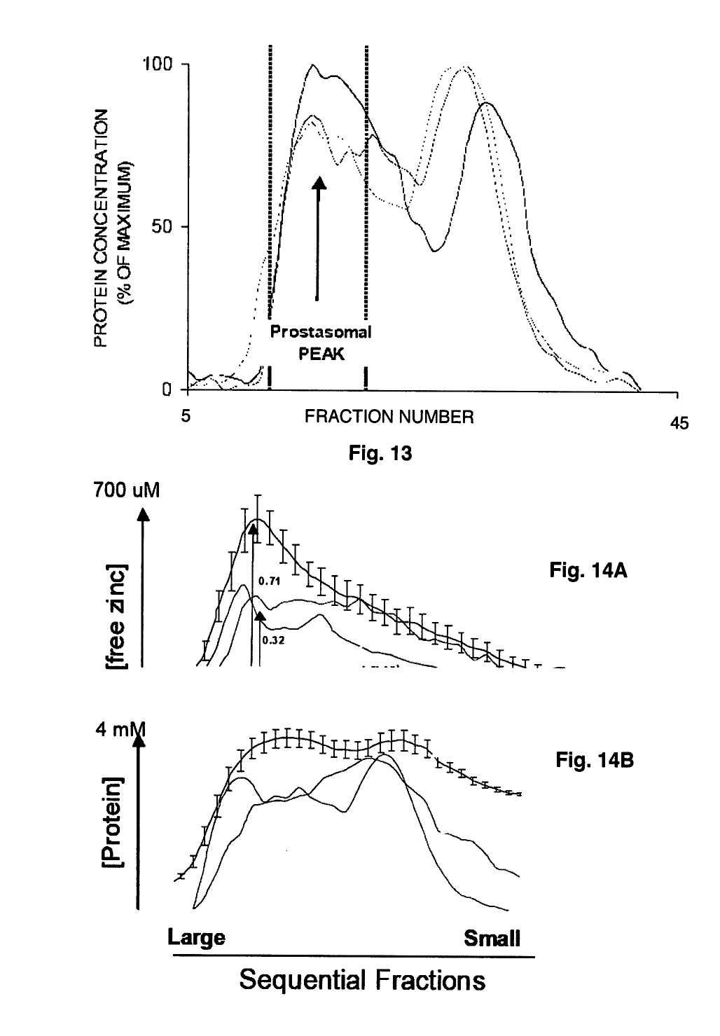

FIG. 13 shows that the seminal protein has two distinct peaks, one early peak that corresponds to the high molecular weight (HMW) proteins and one later low molecular weight (LMW) peak. The HMW peak is confirmed to be highly enriched in the giant globules of prostate-secreted proteins, prostasomes. Thus, the free and toal zinc that was measured from this prostasomal fraction represents the zinc in prostatic fluid per se. The free zinc, which is emblematic of prostatic secretion, is highly enriched in the prostasomal fraction.

Seminal Zinc is Reduced in Gleason Stage 6-8 Tumors

Analysis of the protein and zinc content of seminal plasma demonstrates that men with prostate cancer, confirmed by biopsy, have measurably lower protein and zinc in the prostasomal fraction of seminal fluid. FIGS. 14A-14B shows the total protein measured in each fraction for 15 “normal” men (blue) and 2 men with Gleason stage 7-10 tumors (red). The total protein measured in the seminal plasma of the “normal” men displays the two peaks discussed above, the HMW “prostasomal fraction” and the LMW peak. The peaks are less distinct in the pooled data because fraction numbers are not adjusted to “synchronize” the first peak. The two men with confirmed prostate cancer also have the LMW protein concentration peak but the prostasomal protein fraction peak is essentially absent.

The free zinc in the prostasomal fraction also is markedly lower in both men with cancer (FIG. 14A) and is no more than 50% of the control value. Translating the absorbance measurements to actual concentrations of free zinc, the 0.71 absorbance (baseline substracted) is equal to 2 micromolar in the cuvette; correcting for the dilution (1000-fold) this gives a peak concentration of the free zinc in the prostasomal fraction of 2 mM in the healthy men and less than half, i.e., 1 mM, for the two cancer patients.

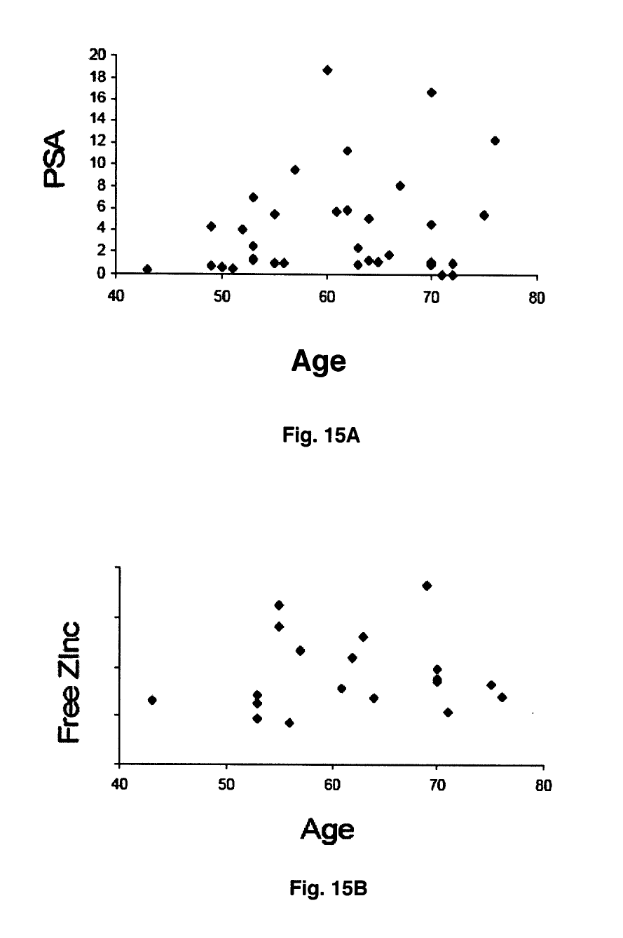

In comparing PSA with age (FIG. 15), only the slightest trend of PSA increasing with age is seen. This is because the men who would have very low PSA (under 40) have not been tested. To evaluate whether prostasomal zinc varies with age or PSA, the average peak concentration of zinc in the prostasomal fraction was calculated. No correlation was found between prostasomal free zinc and either age or PSA (FIG. 15). This indicates that the zinc data are a predictor of prostate cancer independent of PSA. Equally important, the correlation between total zinc concentration in seminal plasma and the concentration of free zinc in the prostasomal fraction was essentially zero (r=0.003). This means that the two measures are not simply redundant estimates of the prostate function.

Cancer Detection Via Free Zinc Analysis in Prostatic Fluid

Post-massage prostatic fluid was collected from men with no prostate disease (n=3) and from one man with a biopsy-confirmed adenocarcinoma. Prostatic fluid was collected by touching off drops of fluid on the end of the penis to a flat surface. The zinc assay was performed on 10 microliters which was added directly to a cuvette containing trichloroacetic acid and one of the calorimetric zinc-sensing chelators that changes absorption or color upon binding and “free” zinc, i.e., zinc that is rapidly exchangeable between the sample matrix and the indicator, 2-(5-bromo-2-pyridylazo-)-5-(N-n-propyl-N-3-sulfopropylamino)phenol. Visual inspection demonstrated that the sample from the man with prostate cancer was qualitatively different in color (data not shown), i.e., about 8-fold differences in absorption, from the samples of the three BPH subjects.

Cancer Prediction from Analysis of Total Zinc in Prostatic Fluid

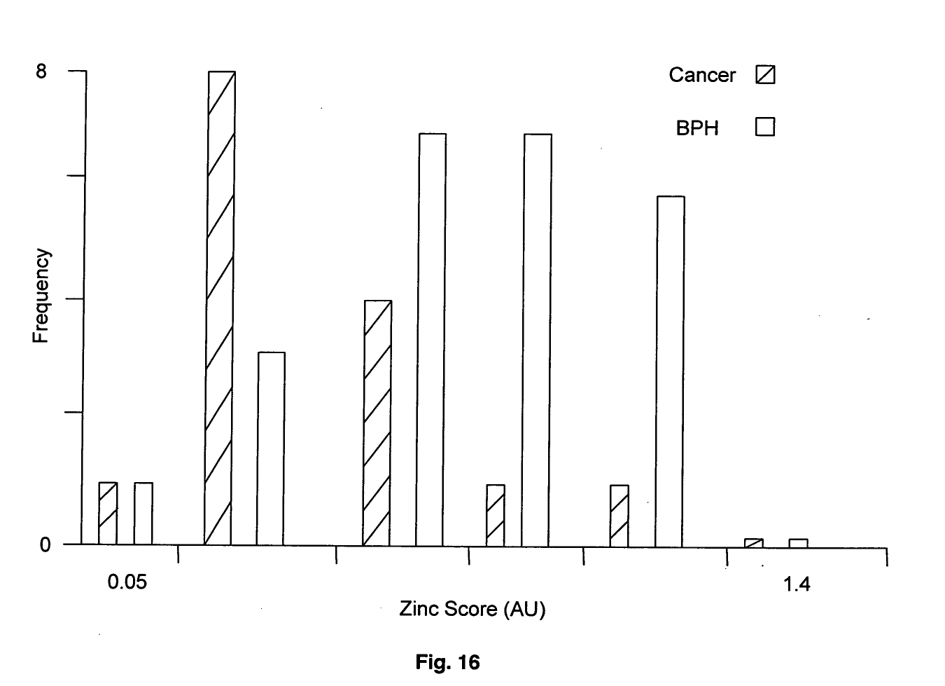

Expressed prostatic fluid was obtained as described previously from 39 men. Later biopsies showed that 15 of the men had adenocarcinoma and 25 had BPH. To assay zinc, the drops of prostatic fluid were placed directly from the urethra onto a filter paper and zinc in the wetted spot was then quantitated by energy-dispersive x-ray fluorescence spectrometry (FIG. 16). By recording emission peaks for iron and zinc demonstrates that the iron peak did not vary appreciably among subjects. Therefore, in calculating the ROC curve, the ratio of Zn/Fe was used as a ratiometric measurement of the zinc.

Despite the small sample size, the resulting area under the curve (AUC) demonstrated 80% specificity for identification of prostate cancer (FIG. 17). This is a better ROC cancer prediction result than is generally obtained by measuring PSA which typically yields an AUROC of only 40%-50%. Thus, measuring zinc, particularly in conjunction with measuring PSA, would significantly increase identification of prostate tumors.

### EXAMPLE 7

Histochemical Imaging of Prostate

These studies address the basic cell biology of zinc in prostate and the commercial goal of imaging the prostate for cancer diagnosis. Results from these studies will give important insights into the fundamentals of zinc metabolism and allow zinc testing of biopsy material to be included as an additional method of diagnosing prostate cancer.

The tissues to be used in this work include prostates harvested from normal men who died without any prostate disease and prostates harvested by prostatectomy or by autopsy from men who had confirmed aggressive prostate cancer. The tissues will be frozen without fixative within an 8-hour postmortem interval. This can include tissues in existing tissue banks, so long as the tissue is frozen without fixative within 0-8 hours postmortem.

Determinations of Tissue Distribution of Total Zinc by Synchrotron-Induced X Ray Fluorescence Imaging

Frozen sections are cut and mounted on glass slides (1 series) and on mylar slides (1 series). The glass-mounted tissue is fixed over aldehyde vapor, then in aldehyde solution for conventional immunostaining to identify various prostate cytoarchitectonic regions. The mylar-mounted sections are sealed in dust-free containers and then sent for processing by synchrotron-induced X Ray fluorescence imaging.

Distribution of Free Zinc at the Macroscopic and Light Microscopic Level

Fresh-frozen tissue sections are stained with either TSQ or Newport Green (cell permeable) or Zinpyr for imaging of the intracellular zinc pools. It is worth noting that the different stains show different “pools” of zinc in the tissue. Thus, for example, the lipophilic stains (TSQ and Zinpyr) will readily stain zinc that is sequestered in the secretory granules or zincosomes in which it is most highly concentrated. Newport green and apoCA-ABDN, on the other hand, will vividly stain cytoplasmic zinc (24) but cannot penetrate these zincosomes and will not stain those cell compartments. Thus, comparison of the differences in staining would indicate subcellular localization of zinc.

Localization of Zinc at the High-Magnification Light and Electron-Microscopic Level

The silver methods of Danscher are the only method of choice. For the silver staining or autometalography (AMG), the tissue is sectioned frozen, then exposed to sulphide vapor (HS) while kept frozen. This treatment precipitates zinc as ZnS in the frozen tissue, thus immobilizing it in situ in whatever subcellular organelles it happens to be. After sulphide precipitation, the tissue is fixed by further exposure to aldehyde vapor (still frozen) before conventionally fixed by aldehyde immersion. Next the tissue sections are developed in a silver developer solution in which the ZnS crystals catalyze reduction of silver, forming silver nanoparticles around the ZnS. Developed sections can then be either counter-stained, cleared, and cover-slipped for light microscope analysis; or dehydrated, embedded in plastic, and ultratomed for analysis in electron microscope.

The following references were cited herein:
1. NCI Fact Sheet 5.29, Jan. 11, 2001, on cis.nci.gov of Natl Cancer Inst.
2. Zaichick et al., Int. Urol. Nephrol., 28:687-694 (1996).
3. Costello and Franklin, Oncology, 59:269-282 (2000).
4. Brys et al., Biol. Trace Elem. Res., 59:145-152 (1997).
5. McCallum et al., Br. J. Urol., 62:565-570 (1988).
6. Ogunlewe and Osegbe, Cancer 63:1388-1392 (1989).
7. Wong et al., Tumour Biol., 21:328-336 (2000).
8. Wilden and Robinson, Br. J. Urol., 47:295-299 (1975).
9. Chang et al., PNAS, 101(5):1129-1134 (2004).
10. Benters et al., Biochem. J., 322:793-9 (1997).
11. Gold and Forber, Investigative Urol., 8:231-238 (1970).
12. Kristiansen et al., Histochem. Cell Biol., 11:125-129 (2001).
13. Danscher, Histochemistry, 71:1-16 (1981).
14. Klitenick et al., Analytical Chemistry 55:921-923 (1983).
15. Frederickson et al., Brain Research, 273:335-339 (1983).
16. Thompson et al., JNeurosci. Meth., 96:35-45 (2000).
17. Li et al., J. Neuroscience (2001).
18. Brown et al., Fertil Steril., 64:612-622 (1995).
19. Moore, J Polymer Sci., A2:835-843 (1964).
20. Fierke and Thompson, Biometals., 141:205-222 (2001).
21. Huang et al., Biochemistry 35:3439-3446 (1996).
22. Frederickson et al., J. Chem. Neuroanat., 5:521-530 (1992).
23. Gao et al., Analyst, 127:1700-4 (2002).
24. Suh et al., J. Histochem. Cytochem., 47:969-972 (1999).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for screening an individual at risk for prostate cancer, comprising:
obtaining a sample of a zinc-containing fluid from the individual;
measuring a level of free zinc and/or bound zinc in the sample;
comparing the zinc level(s) from the at risk individual with zinc levels found in a normal individual known not to have prostate cancer; and
correlating a decreased zinc level in the at-risk individual compared to the level of free zinc in the normal individual to a risk of developing prostate cancer, thereby screening the individual.

2. The method of claim 1 wherein the measured zinc level is the free zinc in the fluid or a ratio of the free zinc to the bound zinc.

3. The method of claim 1, further comprising:
measuring the total protein in the sample.

4. The method of claim 3, wherein measuring the total amount of protein in the fluid comprises measuring ultraviolet light absorption of the protein in the sample.

5. The method of claim 1 wherein the measured zinc level is a ratio of the free zinc to the total protein, a ratio of the bound zinc to the total protein, or a ratio of free zinc plus bound zinc to the total protein.

6. The method of claim 1, wherein the level of free zinc in the fluid is measured optically, the method comprising:
exposing the fluid to a chromophore or fluorophore each comprising a zinc-binding moiety;
binding the free zinc to the zinc-binding moiety of the fluorophore or chromophore;
illuminating the fluid with light;
measuring the amount of light that is either absorbed by the chromophore or emitted by the fluorophore; and
correlating a change in light absorption or light emission with the level of free zinc in the fluid.

7. The method of claim 6, wherein the chromaphore is dithizone, zincon, 4-(2-pyridylazo)resorcinol or other chromaphore that changes absorptive properties upon binding zinc.

8. The method of claim 6, wherein the fluorophore is fluorescein, rhodamine, allexa, or dansylamide.

9. The method of claim 6, wherein the zinc-binding moiety is quinoline, BAPTA, ethylene diamine tetra acetic acid, pyridine, or TPEN.

10. The method of claim 6, further comprising:
releasing the zinc bound to endogenous ligands in the fluid as free zinc; and
measuring a level of free zinc electrochemically before and after releasing the bound zinc.

11. The method of claim 10, further comprising:
measuring total free zinc fluorimetrically or absorptiometrically.

12. The method of claim 10, wherein the free zinc is separated from the fluid via a membrane semi-permeable to zinc.

13. The method of claim 10 wherein the free zinc is separated from the fluid by placing the fluid against a surface containing carrier or iontophore molecules effective to transport zinc ions across the surface.

14. The method of claim 6, further comprising:
releasing the zinc bound to endogenous ligands in the fluid as free zinc; and
measuring a level of free zinc before and after releasing the bound zinc, said free zinc separated from the fluid comprising the endogenous ligands.

15. The method of claim 1, wherein the zinc-containing fluid comprises whole ejaculate, whole seminal fluid, seminal plasma, or prostatic fluid.

16. A method for screening an individual at risk for prostate cancer, comprising:
obtaining a sample of prostate secretions in a fluid from the individual;
measuring a level of free zinc in the fluid sample;
comparing the level of free zinc from the at risk individual with a level of free zinc in a normal individual that does not have prostate cancer; and
correlating a decreased level of free zinc in the at-risk individual compared to the level of free zinc in the normal individual to a risk of developing prostate cancer, thereby screening the individual.

17. The method of claim 16, wherein the prostate secretions are in a fluid comprising seminal plasma of whole ejaculate, the step of obtaining comprising:
separating large globular proteins and prostasomes from the seminal plasma including free zinc via size-exclusion column fractionation.

18. The method of claim 16, wherein the prostate secretions are in a fluid comprising seminal plasma of whole ejaculate, the step of obtaining comprising:
separating large globular proteins and prostasomes from the seminal plasma including free zinc via antibody- or aptamer-binding thereto.

19. The method of claim 16, wherein the prostate secretions are in prostatic fluid, the step of obtaining comprising:
massaging the prostate to advance the prostatic fluid comprising the prostate secretions into the uretha; and
collecting a post prostatic massage prostatic fluid therefrom.

20. The method of claim 19, wherein the prostatic fluid is collected from the urethra in a first volume of urine produced post prostatic massage.

21. The method of claim 19, further comprising:
repeating massage until the prostatic fluid emerges from the urethra; and
collecting the post prostatic massage prostatic fluid onto a surface.

22. The method of claim 16, wherein free zinc in the prostatic fluid is measured fluorimetrically using a fluorophore comprising a zinc-binding moiety.

23. The method of claim 22, wherein the fluorophore is fluorescein, rhodamine, allexa, or dansylamide.

24. The method of claim 22, wherein the zinc-binding moiety is quinoline, BAPTA, ethylene diamine tetra acetic acid, pyridine, or TPEN.

25. The method of claim 22, further comprising:
adding detergent to the prostatic fluid; and
lysing and dissociating prostasomes and globular proteins in the prostatic fluid to release zinc bound thereto as free zinc; wherein free zinc is measured before and after lysing.

25. The method of claim 22, further comprising:
mixing the fluorophore with the prostatic fluid containing the free zinc.

26. The method of claim 22, further comprising:
attaching the fluorophore at a distance no more than 350 nm from a surface of a solid substrate to which the prostatic fluid containing the free zinc is exposed.

27. The method of claim 26, further comprising:
exciting the fluorophore with an evanescent wave of light that is totally internally reflected within the solid substrate; and
detecting the light emissions of the excited fluorophore to measure the free zinc.

28. The method of claim 26, further comprising:
positioning a sensor on a surface of the solid substrate opposite to the surface exposed to the prostatic fluid to detect fluorescent emissions of a light excited fluorophore to measure the free zinc.

29. The method of claim 22, further comprising:
separating the prostatic fluid from the fluorophore via a semipermeable membrane permeable to zinc ions but not permeable to the fluorophore.
